(19) United States
(12) Patent Application Publication
(10) Pub. No.: US 2008/0215055 A1
(43) Pub. Date: Sep. 4, 2008

(54) Method and apparatus for a planar drill

(75) Inventor: Kevin T. Stone, Winona Lake, IN (US)

Correspondence Address: HARNESS, DICKEY & PIERCE, P.L.C., P.O. BOX 828, BLOOMFIELD HILLS, MI, 48303, US

(73) Assignee: Kevin T. Stone

(21) Appl. No.: 11/713,112

(22) Filed: Mar. 1, 2007

(51) Int. Cl. A61B 17/58

(52) U.S. Cl. 606/80

(57) ABSTRACT

A method of preparing a surface of a joint for receipt of a graft is provided. The method can include providing a cutting tool having a solid, shallow angle cutting surface. The method can also include cutting a shallow section out of the anatomy with the solid, shallow angle cutting surface to form a bore.

### FIELD

The present teachings relate generally to surgical instruments and procedures, and particularly to a method and apparatus for a planer drill.

### BACKGROUND

The statements in this section merely provide background information related to the present disclosure and may not constitute prior art.

Many portions of the human anatomy naturally articulate relative to one another. Generally, the articulation between the portions of the anatomy is substantially smooth and without abrasion. This articulation is allowed by the presence of natural tissues, such as cartilage and strong bone.

Over time, however, due to injury, stress, health issues and various other issues, articulation of the various portions of the anatomy can become rough or impractical. For example, injury can cause the articular cartilage coupled to the boney structure and/or boney structure to become damaged, resulting in an osteochondral lesion. Osteochondral lesions can impair the articulation of the particular fractured anatomical portion.

At such times, it can be desirable to repair the damaged anatomical portion with a graft or replacement device such that normal or easy articulation can be reproduced. Typically, a bore may need to be formed in the anatomical portion to enable the attachment of the graft. Generally, these bores may be formed by using a sharp angle cutting tool that, over one rotation, quickly removes large sections of the boney structure. In addition, these cutting tools tend to not have a peripheral cutting edge, which can damage the surrounding tissue. In some instances, the quick removal of the boney structure may result in trauma to the surrounding tissue due to the coarse nature of the sharp angled cutting tool. In addition, these cutting tools tend to not have a peripheral cutting edge, which can damage the surrounding tissue. In addition, as the sharp angled cutting tool removes large portions of the boney structure per revolution, it may be hard to control the depth of the bore formed with the sharp angled cutting tool. Furthermore, the coarse nature of the cutting performed by the sharp angled cutting tool may reduce visibility during the formation of the bore, which may also result in bores that are deeper than desired. Therefore, it may be desirable to provide a planar drill for forming a bore in an anatomical portion for receipt of a graft in which the planar drill bit is capable of forming the bore by removing smaller, more controlled sections of the boney structure with reduced potential trauma to the surrounding tissue.

### SUMMARY

A method of preparing a portion of an anatomy for receipt of a graft. The method can include providing a cutting tool having a solid, shallow angle cutting surface. The method can also include cutting a shallow section out of the anatomy with the solid, shallow angle cutting surface to form a bore.

A method of preparing a portion of an anatomy for receipt of a graft is provided. The method can include providing a cutting tool having a solid, shallow angle cutting surface or a shallow angle cutting surface with a peripheral surface portion and providing a graft or graft substitute. The method can also include cutting a shallow section out of the anatomy with the solid, shallow angle cutting surface to form a bore. The method can include inserting the graft or graft substitute into the bore.

Also provided is a method of preparing a portion of an anatomy for receipt of a graft. The method can include providing a cutting tool having a solid, shallow angle cutting surface, the shallow angle of the cutting surface ranging from 10 to 45 degrees. The method can also include providing the cutting tool with a central cutting surface having a defined cutting surface with the defined cutting surface selected from the group comprising a flat cutting surface, a concave cutting surface, and a convex cutting surface. The method can include providing a graft. The method can also include cutting a shallow section out of the joint with the solid, shallow angle cutting surface to form a bore. The method can include forming a bore with a contoured bottom surface. The contour of the bottom surface can be substantially similar to the defined cutting surface of the central cutting surface. The method can further include inserting the graft into the bore.

Further areas of applicability will become apparent from the description provided herein. It should be understood that the description and specific examples are intended for purposes of illustration only and are not intended to limit the scope of the present disclosure.

### DRAWINGS

The drawings described herein are for illustration purposes only and are not intended to limit the scope of the present disclosure in any way.

FIG. 1 is a perspective view of a planer cutting tool according to various embodiments;

FIG. 2 is a top view of the planer cutting tool of FIG. 1;

FIG. 3 is a side view of the planer cutting tool of FIG. 1;

FIG. 4 is an environmental view of a procedure for using the planer cutting tool of FIG. 1 on a first portion of an anatomy;

FIG. 5 is a cross-sectional environmental view of the procedure of FIG. 4, taken along line 5-5 of FIG. 4;

FIG. 6 is an environmental view of a bore formed by the planer cutting tool of FIG. 1 on the first portion of the anatomy;

FIG. 7 is a perspective view of an alternative planer cutting tool according to various embodiments;

FIG. 8 is a top view of the alternative planer cutting tool of FIG. 7;

FIG. 9 is a side view of the alternative planer cutting tool of FIG. 7;

FIG. 10 is a partial cross-sectional environmental view of a procedure for using the alternative planer cutting tool of FIG. 9 on a portion of an anatomy;

FIG. 11 is a cross-sectional environmental view of the procedure of FIG. 10, taken along line 11-11 of FIG. 10;

FIG. 12 is an environmental view of a bore formed by the alternative planer cutting tool of FIG. 7 on the portion of the anatomy;

FIG. 13 is a perspective view of a second alternative planer cutting tool according to various embodiments;

FIG. 14 is a top view of the second alternative planer cutting tool of FIG. 13;

FIG. 15 is a side view of the second alternative planer cutting tool of FIG. 13;

FIG. 16 is a partial cross-sectional environmental view of a procedure for using the second alternative planer cutting tool of FIG. 13 on a portion of an anatomy;

FIG. 17 is a cross-sectional environmental view of the procedure of FIG. 16, taken along line 17-17 of FIG. 16;

FIG. 18 is an environmental view of a bore formed by the second alternative planer cutting tool of FIG. 13 on the portion of the anatomy.

FIG. 19 is a perspective view of a third alternative planer cutting tool according to various embodiments;

FIG. 20 is a top view of the third alternative planer cutting tool of FIG. 19;

FIG. 21 is a side view of the third alternative planer cutting tool of FIG. 19;

FIG. 22 is an environmental view of a procedure for using the third alternative planer cutting tool of FIG. 19 on a portion of an anatomy;

FIG. 23 is a cross-sectional environmental view of the procedure of FIG. 23 taken along line 23-23 of FIG. 22;

FIG. 24 is a perspective view of a fourth alternative planer cutting tool according to various embodiments;

FIG. 25 is a top view of the fourth alternative planer cutting tool of FIG. 24;

FIG. 26 is a side view of the fourth alternative planer cutting tool of FIG. 24;

FIG. 27 is an environmental view of a procedure for using the fourth alternative planer cutting tool of FIG. 24 on a portion of an anatomy; and

FIG. 28 is a cross-sectional environmental view of the procedure of FIG. 27, taken along line 28-28 of FIG. 27.

### DETAILED DESCRIPTION

The following description is merely exemplary in nature and is not intended to limit the present disclosure, application, or uses. Although the following description is related generally to an apparatus for forming a bore in a portion of the anatomy, such as through the articular cartilage and distal end of a femur of a knee joint, with a planer drill, it will be understood that the method and apparatus for a planer drill, as described and claimed herein, can be used with any appropriate surgical procedure, and with any boney structure. Therefore, it will be understood that the following discussions are not intended to limit the scope of the appended claims.

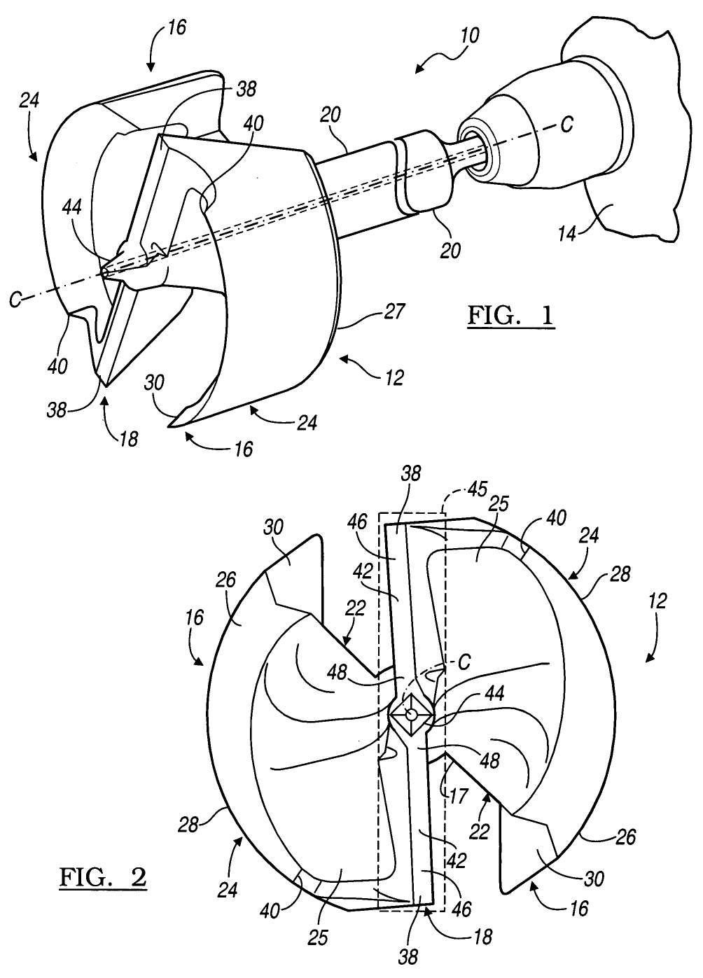

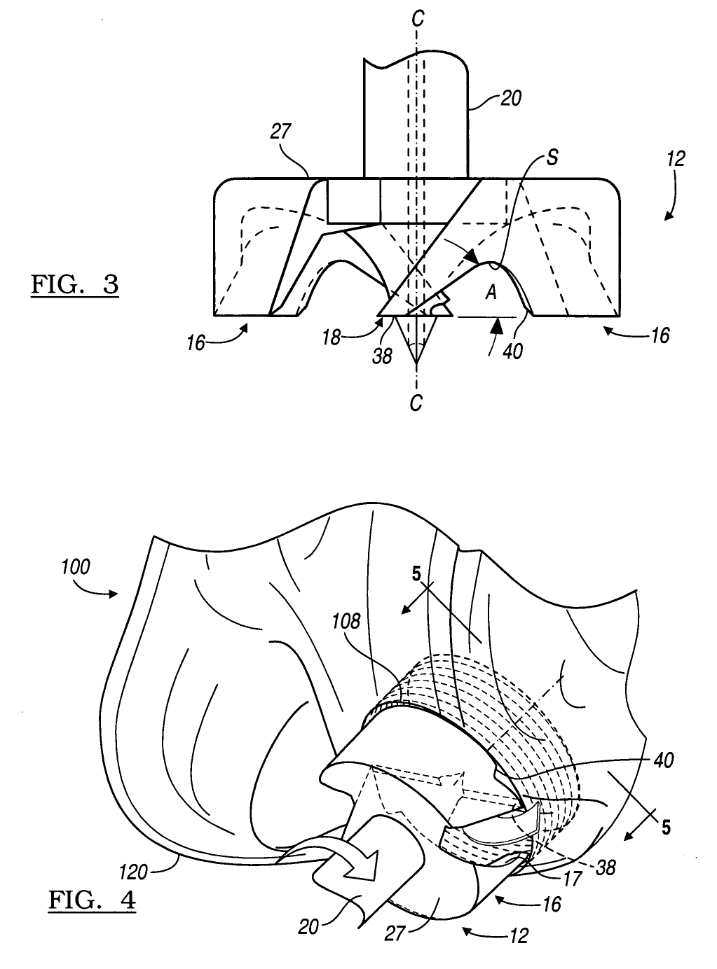

As will be discussed in more detail herein, an apparatus for a planer drill 10 is disclosed. With reference to FIG. 1, the planer drill 10 can include a planer drill bit 12 which can be coupled to an appropriate tool, such as a drill 14. As the drill 14 can operate in a generally known manner, a detailed discussion of the components and operation of the drill 14 need not be provided herein. With additional reference to FIG. 2, the drill bit 12 can include at least one peripheral cutting portion 16, a central cutting portion 18 and a shaft 20. The peripheral cutting portion 16 can be coupled to the central cutting portion 18, and the peripheral cutting portion 16 and the central cutting portion 18 can be coupled to the shaft 20. The shaft 20 can couple the drill bit 12 to the drill 14. The shaft 20 can also define a throughbore for receipt of a guide wire to guide the drill bit 12 into an anatomy (not shown). Generally, the peripheral cutting portion 16 and central cutting portion 18 can define a solid, shallow angle cutting surface to form a bore with vertical sidewalls in a portion of the anatomy, as will be described herein.

Typically, the drill bit 12 can include two peripheral cutting portions 16. The peripheral cutting portions 16 can be composed of a metal or metal alloy material with sufficient rigidity to cut through the desired portion of the anatomy. The peripheral cutting portions 16 can generally be arcute or semi-circular about a centerline C of the drill bit 12. An angular channel 17 can be disposed between each of the peripheral cutting portions 16 to enable cut sections of an anatomy to be removed from a cutting path as will be discussed in greater detail herein. Generally, the angular channel 17 between the peripheral cutting portions 16 can range from 0 to 60 degrees, and can typically range from 10 to 25 degrees.

The peripheral cutting portions 16 can each include a base 22 and a sidewall 24. The base 22 can include a generally concave interior surface 25 and an exterior surface 27. The concave interior surface 25 can provide an area for channeling the cut sections of the anatomy (not specifically shown). The exterior surface 27 can couple the peripheral cutting portions 16 to the shaft 20 (not specifically shown). It should be noted that the peripheral cutting portions 16 can be integrally formed with the shaft 20, or the peripheral cutting portions 16 could be coupled to the shaft 20 via welding, mechanical fasteners and the like. The sidewall 24 can be coupled to or integrally formed with the base 22.

The sidewall 24 can include a first end 26 and a second end 28. The first end 26 can define a tapered surface 30. The tapered surface 30 can facilitate the channeling of the cut sections of the anatomy. The second end 28 can include a first cutting surface 38 and a second cutting surface 40. The first cutting surface 38 can generally be formed with a shallow cutting angle A so that the first cutting surface 38 can remove only a small amount of the anatomy for each revolution of the drill bit 12. The shallow cutting angle A can range from about 0.01 to 65 degrees and generally from about 20 to 35 degrees. The first cutting surface 38 can also be used to guide the drill bit 12 into the anatomy, as will be discussed in greater detail herein.

The second cutting surface 40 can be disposed adjacent to the first cutting surface 38. The second cutting surface 40 can trail the first cutting surface 38 in the rotation of the drill bit 12 to remove any spurs formed during the cutting performed by the first cutting surface 38, as will be described herein. Generally, the second end 28 of the peripheral cutting surface 16 can have a slope S from the first cutting surface 38 to the second cutting surface 40, to facilitate the removal of the cut sections of anatomy from the first cutting surface 38, but the second end 28 could be planar. The second end 28 can be coupled to or integrally formed with the central cutting portion 18.

The central cutting portion 18 can include at least one or a plurality of central cutting surfaces 42 and a center point 44. The central cutting portion 18 can be coupled to the first cutting surface 38 of the second end 28 of the peripheral cutting portion 16 via the central cutting surfaces 42 to form a solid or uniform cutting surface 45. For example, the central cutting portion 18 can include two central cutting surfaces 42. Each of the central cutting surfaces 42 can include a first end 46 and a second end 48. The first end 46 can be coupled to a respective first cutting surface 38 of the peripheral cutting portions 16 and the second end 48 can be coupled to the center point 44 to form the uniform cutting surface 45. The uniform cutting surface 45 can have a contoured or defined cutting surface characterized by the shape or contour of the central cutting surfaces 42. The central cutting surfaces 42 can cut or shave off an interior portion of the anatomy to form a contoured bore in the anatomy, as will be discussed further herein. The central cutting surfaces 42 can be generally planar, such that central cutting surfaces 42 can form a flat-bottomed bore in the anatomy, as will be discussed herein.

The center point 44 of the central cutting portion 18 can be generally similar to a pyramid in shape, however, any shape could be used. The center point 44 can generally extend beyond the uniform cutting surface 45 to enable the drill bit 12 to be located on the anatomy.

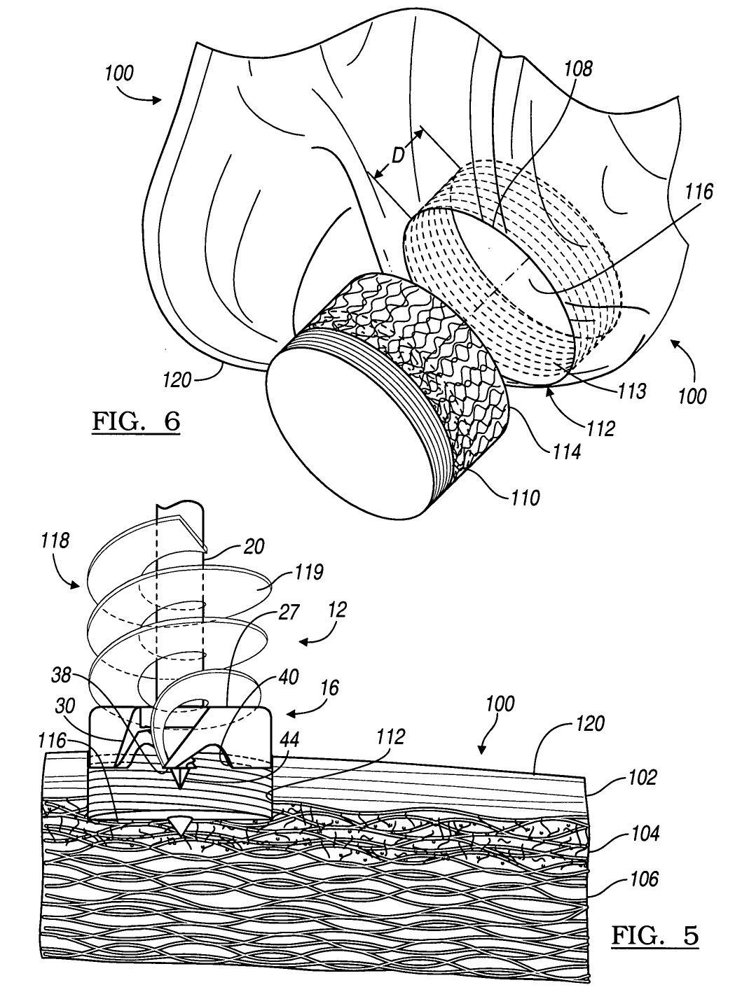

With reference to FIGS. 4, 5 and 6, a selected portion of an anatomy 100 is shown. The anatomy 100 can include a cartilage layer 102, a cortical bone layer 104 and a cancelleous bone layer 106 (FIG. 5). The drill bit 12 can be used to prepare the anatomy 100 for receipt of a graft 110 by forming a bore 112 in the anatomy 100 (FIG. 6). The graft 110 can be an autologous graft, an allograft or a xenograft. Alternatively, a graft substitute could be used, such as a bone substitute material forming a plug, with the bone substitute material comprising polylactide (PLA), polyglycolic acid (PGA), Calcium Phosphate, Calcium Sulfate, TriCalcuim Phosphate or combinations thereof, sized to be received in the bore 112. The graft 110 can have a bottom surface 114 that can be configured to match a bottom surface 116 of the bore 112. Generally, the drill bit 12 can form the bore 112 with the bottom surface 116 having a contour substantially similar to the contour or defined cutting surface of the uniform cutting surface 45 due to a cutting path 118 created by the central cutting surfaces 42.

As shown in FIGS. 4 and 6, the anatomy 100 can include a convex surface 120, such as a distal end of a femur. In order to form the bore 112 in the convex surface 120, the drill bit 12 can be coupled to the drill 14. Next, the drill bit 12 can be positioned adjacent to the anatomy 100 and the drill 14 can be actuated such that the drill bit 12 begins to rotate. As the drill bit 12 begins to rotate, the first cutting surface 38 can guide the drill bit 12 into the anatomy 100. The drill bit 12 can be rotated at any desired speed, such as 60 to 600 revolutions per minute (rpms).

As the drill bit 12 rotates, the uniform cutting surface 45 can remove thin layers 119 of the cartilage layer 102 of the anatomy 100. Generally, the thickness of the layer 119 removed per revolution of the drill bit 12 is between 0.1 mm and 0.5 mm. The first cutting surfaces 38 can cut a perimeter 108 of the bore 112 in the cartilage layer 102, with the central cutting surfaces 42 cutting an interior of the bore 112 in the cartilage layer 102 as shown by the cutting path 118 (FIG. 5). The thin layers 119 of the cartilage layer 102 can pass into the angular channel 17 of the drill bit 12 to remove the cartilage layer 102 from the uniform cutting surface 45. The second cutting surfaces 40 on the peripheral cutting portions 16 can follow the first cutting surfaces 38 to remove any spurs formed by the first cutting surfaces 38 to create the bore 112 with substantially vertical sidewalls 113. The shallow cutting angles A of the first cutting surfaces 38 can enable the drill bit 12 to remove thin layers 119 of the anatomy 100 during each rotation of the drill bit 12, which can be less traumatic to tissue surrounding the bore 112, as shown by the cutting path 118 of the drill bit 12.

The drill bit 12 can be rotated until the drill bit 12 has created the bore 112 with a desired depth D, which could include removing a portion of either the cortical bone layer 104 and/or the cancelleous bone layer 106. After the bore 112 has been formed with the desired depth D, then the graft 110 can be inserted into the bore 112 as shown in FIG. 6.

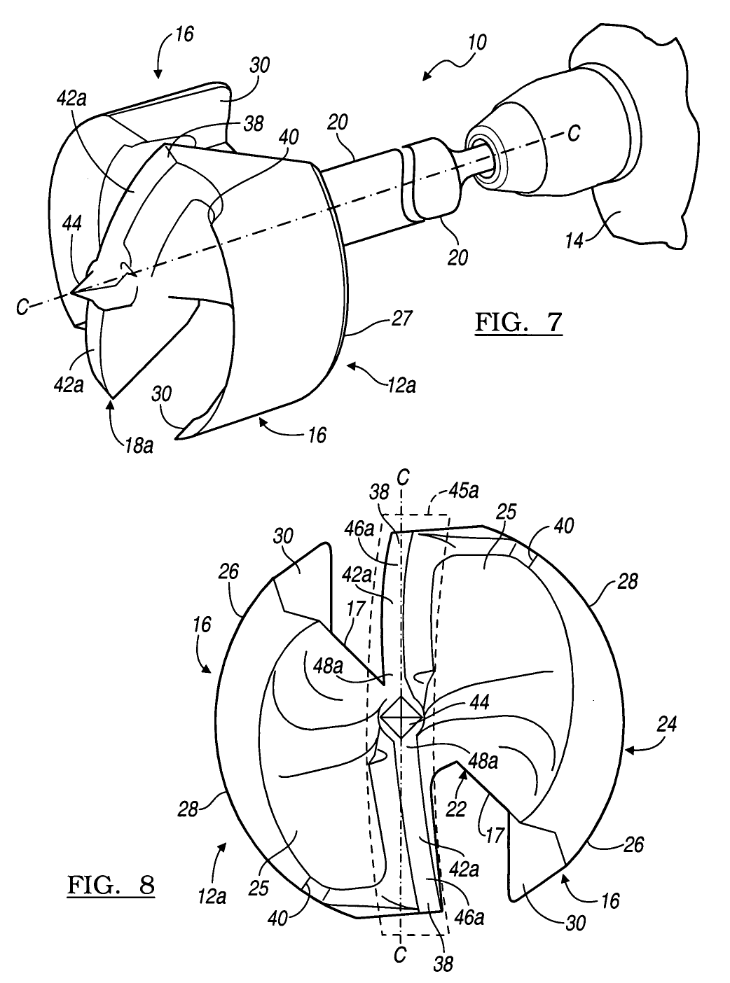

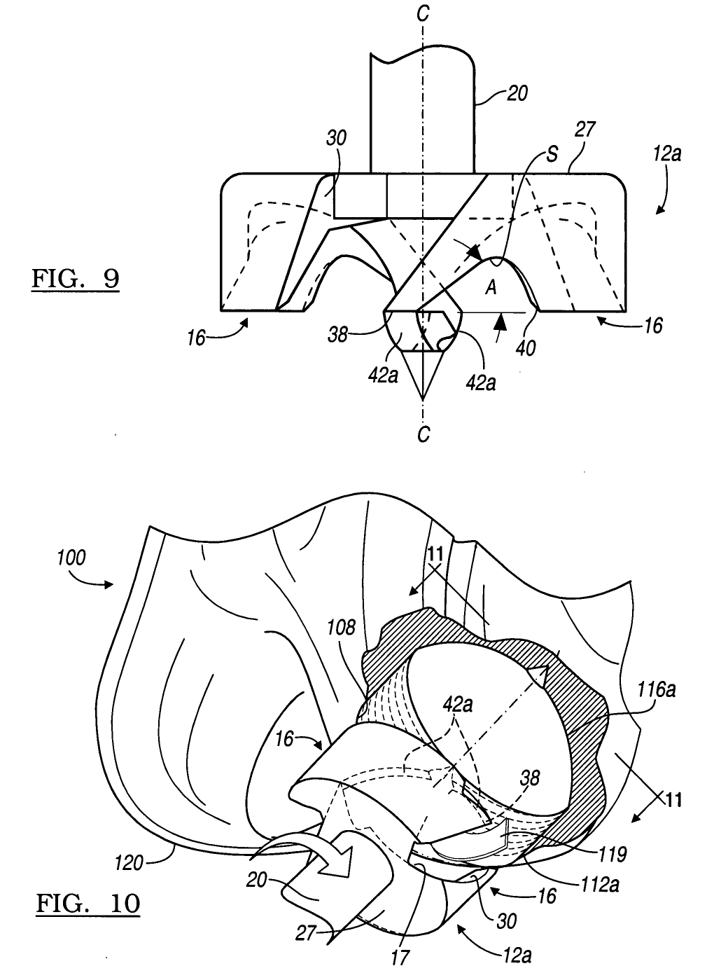

Alternatively, with reference to FIGS. 7, 8 and 9, an alternative drill bit 12a is shown. The drill bit 12a can include the peripheral cutting portion 16, a central cutting portion 18a and the shaft 20. As the peripheral cutting portion 16 and shaft 20 of the drill bit 12a are substantially similar to the peripheral cutting portion 16 and shaft 20 of the drill bit 12, the peripheral cutting portion 16 and shaft 20 will not be discussed in detail with regard to the drill bit 12a.

The central cutting portion 18a can include at least one or a plurality of central cutting surfaces 42a and the center point 44. As the center point 44 of the drill bit 12a is substantially similar to the center point 44 of the drill bit 12, the center point 44 will not be discussed in detail with regard to the drill bit 12a. The central cutting portion 18a can be coupled to the first cutting surface 38 of the second end 28 of the peripheral cutting portion 16 via the central cutting surfaces 42a to form a solid or uniform cutting surface 45a, as discussed with regard to the drill bit 12. The first ends 46a of each of the central cutting surfaces 42a can be coupled to a respective first cutting surface 38 of the peripheral cutting portions 16 and the second ends 48a of each of the central cutting surfaces 42a can be coupled to the center point 44. The central cutting surfaces 42a can be generally concave, so that the central cutting surfaces 42a can cut or shave off an interior portion of the anatomy to form a concave bore in the anatomy, as will be discussed herein.

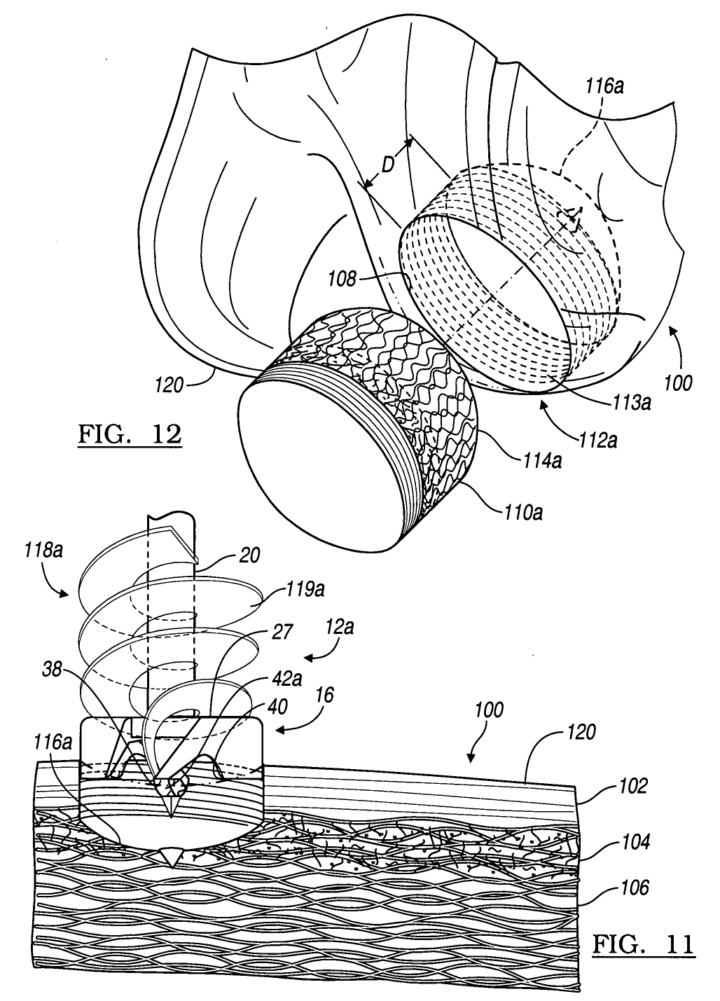

With additional reference now to FIGS. 10, 11 and 12, wherein the same reference numerals denote the same or similar components, the anatomy 100 can include the cartilage layer 102, the cortical bone layer 104 and the cancelleous bone layer 106 (FIG. 11). The anatomy 100 can include the convex surface 120, such as a distal end of a femur. The drill bit 12a can be used form a bore 112a in the anatomy 100 for receipt of a graft 110a. The graft 110a can be an autologous graft, an allograft or a xenograft. Alternatively, a graft substitute could be used, such as a bone substitute material forming a plug, with the bone substitute material comprising polylactide (PLA), polyglycolic acid (PGA), Calcium Phosphate, Calcium Sulfate, TriCalcuim Phosphate or combinations thereof, sized to be received in the bore 112a. As the bore 112a formed by the drill bit 12a can have a generally concave bottom surface 116a, the graft 110a can have a concave bottom surface 114a to correspond with the concave bottom surface 116a of the bore 112a.

In order to form the bore 112a in the anatomy 100, the drill bit 12a can be coupled to the drill 14 (not shown). Next, the drill bit 12a can be positioned adjacent to the anatomy 100 and the drill 14 can be actuated such that the drill bit 12a begins to rotate. As the drill bit 12a begins to rotate, the first cutting surface 38 can guide the drill bit 12a into the anatomy 100.

As the drill bit 12a rotates, the uniform cutting surface 45a can remove thin layers 119a of the cartilage layer 102 of the anatomy 100, with the first cutting surfaces 38 cutting a perimeter 108 of the bore 112a in the cartilage layer 102 and the central cutting surfaces 42a cutting an interior of the bore 112a in the cartilage layer 102. Generally, the thickness of the layer 119a removed per revolution of the drill bit 12a is between 0.1 mm and 0.5 mm. The thin layers 119a of the cartilage layer 102 can pass into the angular channel 17 of the drill bit 12a to remove the cartilage layer 102 from the uniform cutting surface 45a. The second cutting surfaces 40 on the peripheral cutting portions 16 can follow the first cutting surfaces 38 to remove any spurs formed by the first cutting surfaces 38 to create the bore 112a with substantially vertical sidewalls 113a. The shallow cutting angles A of the first cutting surfaces 38 enable the drill bit 12a to remove thin layers 119a of the anatomy 100 during each rotation of the drill bit 12a, which can be less traumatic to tissue surrounding the bore 112a, as shown by the cutting path 118a of the drill bit 12a.

The drill bit 12a can be rotated until the drill bit 12a has created the bore 112a with a desired depth D, which could include removing a portion of either the cortical bone layer 104 and/or the cancelleous bone layer 106. Due to the concave shape of the central cutting surfaces 42a, the bottom surface 116a of the bore 112a can be generally concave. After the bore 112a has been formed with the desired depth D, then the graft 110, (or graft substitute) can be inserted into the bore 112a as shown in FIG. 12.

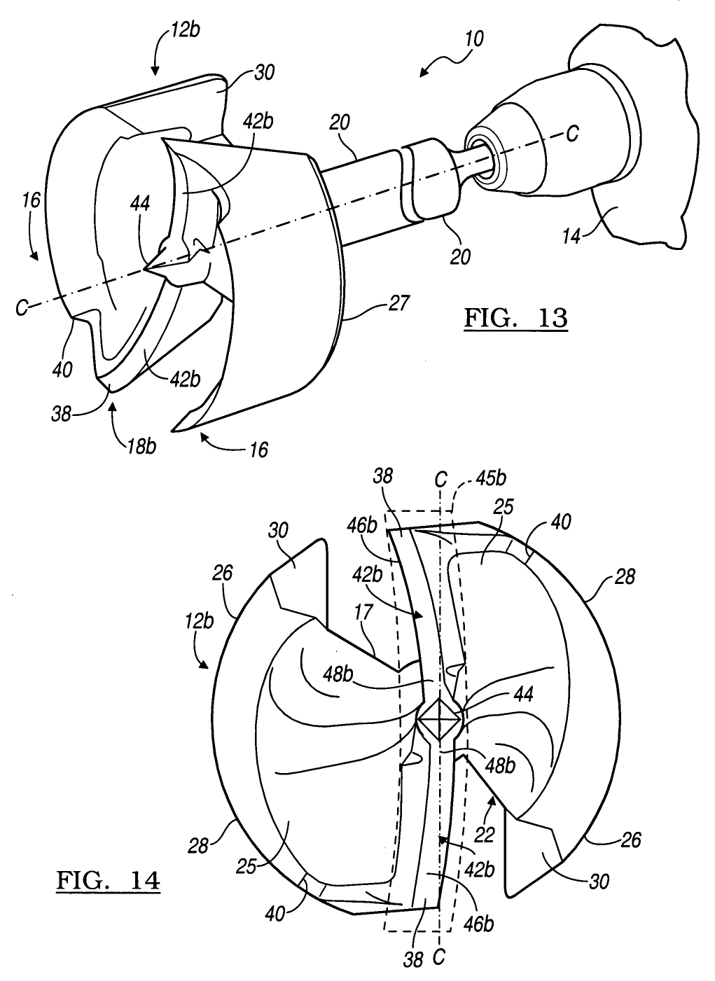

Alternatively, with reference now to FIGS. 13, 14 and 15, a second alternative drill bit 12b is shown. The drill bit 12b can include the peripheral cutting portion 16, a central cutting portion 18b and the shaft 20. As the peripheral cutting portion 16 and shaft 20 of the drill bit 12b are substantially similar to the peripheral cutting portion 16 and shaft 20 of the drill bit 12, the peripheral cutting portion 16 and shaft 20 will not be discussed in detail with regard to the drill bit 12b.

The central cutting portion 18b can include at least one or a plurality of central cutting surfaces 42b and the center point 44. As the center point 44 of the drill bit 12b is substantially similar to the center point 44 of the drill bit 12, the center point 44 will not be discussed in detail with regard to the drill bit 12b. The central cutting portion 18b can be coupled to the first cutting surface 38 of the second end 28 of the peripheral cutting portion 16 via the central cutting surfaces 42b to form a solid or uniform cutting surface 45b, as discussed previously. Each of the central cutting surfaces 42b can include a first end 46b and a second end 48b. Each of the first ends 46b can be coupled to a respective first cutting surface 38 of the peripheral cutting portions 16 and each of the second ends 48b can be coupled to the center point 44. The central cutting surfaces 42b can cut or shave off an interior portion of the anatomy to form a bore in the anatomy, as will be discussed further herein. The central cutting surfaces 42b can be generally concave, so that the central cutting surfaces 42b can form a convex bore in the anatomy.

With additional reference now to FIGS. 16, 17 and 18, wherein the same reference numerals denote the same or similar components, the anatomy 100 can include the cartilage layer 102, the cortical bone layer 104 and the cancelleous bone layer 106. The anatomy 100 can include the concave surface 120, such as a proximal tibia. The drill bit 12b can be used to form a bore 112b in the concave surface 120, for receipt of a graft 110b. The graft 110b can be an autologous graft, an allograft or a xenograft, or combinations thereof, sized to be received in the bore 112b.

In order to form the bore 112b, the drill bit 12b can be coupled to the drill 14. Next, the drill bit 12b can be positioned adjacent to the anatomy 100 and the drill 14 can be actuated such that the drill bit 12b begins to rotate. As the drill bit 12b begins to rotate, the first cutting surface 38 can guide the drill bit 12b into the anatomy 100.

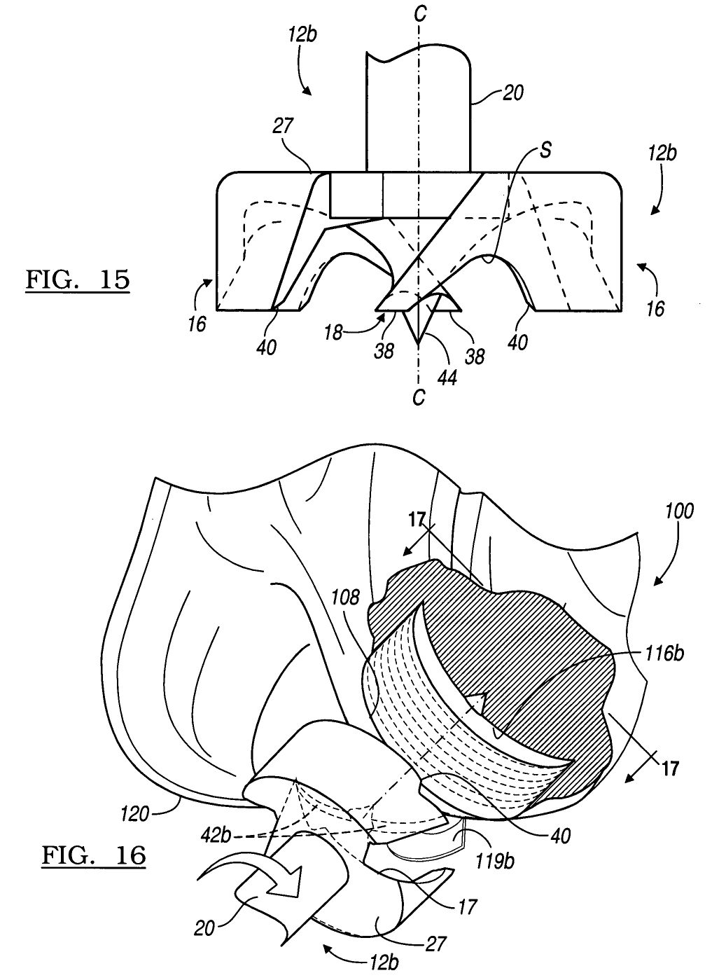

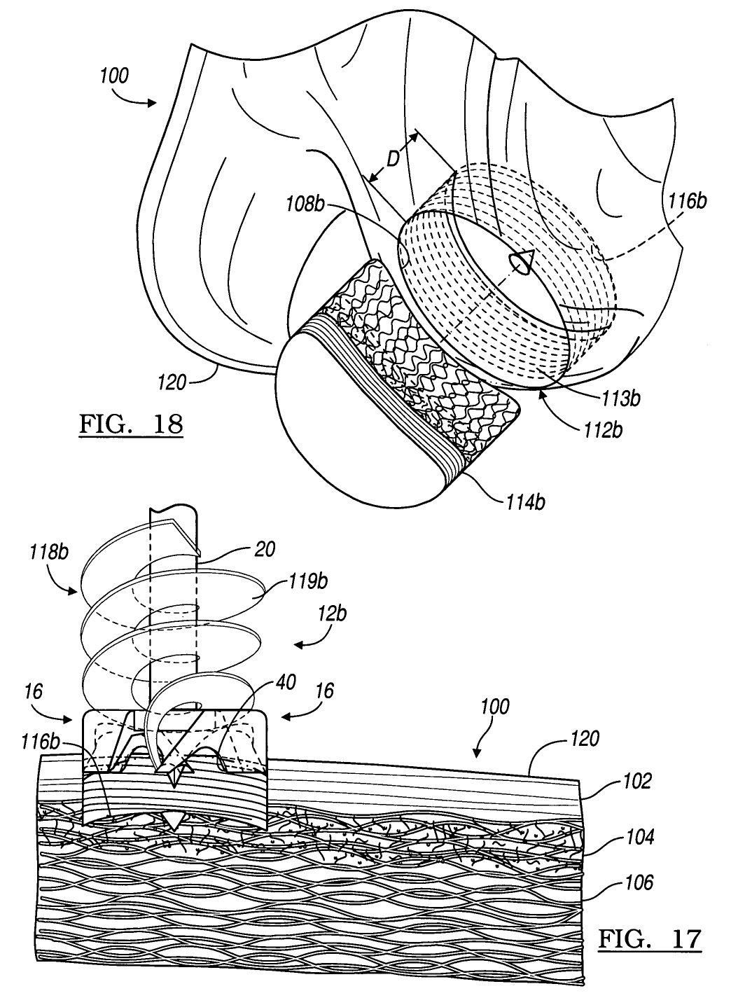

As the drill bit 12b rotates, the uniform cutting surface 45b can remove thin layers 119b of the cartilage layer 102 of the anatomy 100, with the first cutting surfaces 38 cutting a perimeter 108b of the bore 112b in the cartilage layer 102 and the central cutting surfaces 42b cutting an interior of the bore 112b in the cartilage layer 102. Generally, the thickness of the layer 119b removed per revolution of the drill bit 12b is between 0.1 mm and 0.5 mm. Due to the concave shape of the central cutting surfaces 42b, the central cutting surfaces 42b form a bore 112b with a convex bottom surface 116b. The thin layers 119b of the cartilage layer 102 can pass the angular channel 17 of the drill bit 12b to remove the cartilage layer 102 from the uniform cutting surface 45b. The second cutting surfaces 40 on the peripheral cutting portions 16 can follow the first cutting surfaces 38 to remove any spurs formed by the first cutting surfaces 38 to create the bore 112b with substantially vertical sidewalls 113b. The shallow cutting angles A of the first cutting surfaces 38 can enable the drill bit 12b to remove thin layers 119 of the anatomy 100 during each rotation of the drill bit 12b, which can be less traumatic to tissue surrounding the bore 112b, due to the shallow cuts made by the drill bit 12b, due to the shallow cuts made by the drill bit 12b as shown by the cutting path 118b of the drill bit 12b (FIG. 17). After the bore 112b has been formed with the desired depth D, then the graft 110b (or graft substitute) can be inserted into the bore 112b as shown in FIG. 15.

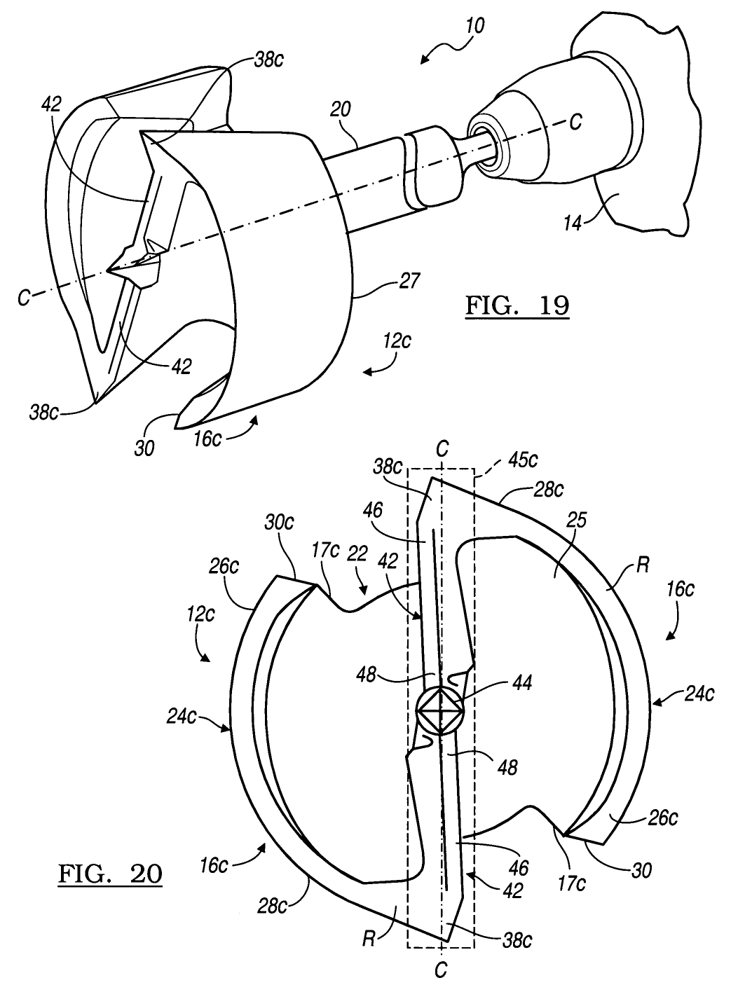

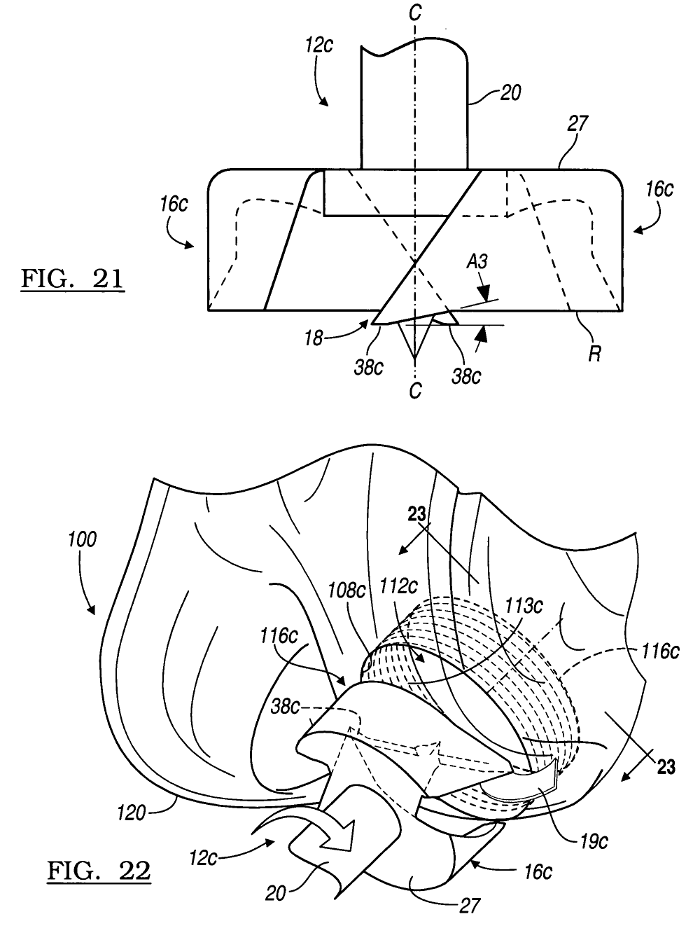

Alternatively, with reference now to FIGS. 19, 20 and 21, a third alternative drill bit 12c is shown. The drill bit 12c can include a peripheral cutting portion 16c, the central cutting portion 18 and the shaft 20. As the central cutting portion 18 and shaft 20 of the drill bit 12c are substantially similar to the central cutting portion 18 and shaft 20 of the drill bit 12, the central cutting portion 18 and shaft 20 will not be discussed in detail with regard to the drill bit 12c. It should be noted that although the drill bit 12c is shown to have the central cutting portion 18 associated with the drill bit 12, the drill bit 12c could also have the central cutting portion 18a associated with the drill bit 12a or the central cutting portion 18b associated with the drill bit 12b.

The peripheral cutting portion 16c of the drill bit 12c can include two peripheral cutting portions 16c. The peripheral cutting portions 16c can be composed of a metal or metal alloy material with sufficient rigidity to cut through the desired portion of the anatomy. The peripheral cutting portions 16c can generally be arcuate or semi-circular about a centerline C of the drill bit 12c. An angular channel 17c can be disposed between each of the peripheral cutting portions 16c to enable cut sections of an anatomy to be removed from a cutting path as will be discussed in greater detail herein. Generally, the angular channel 17c between the peripheral cutting portions 16c can range from 0 to 60 degrees, and typically ranges from 35 to 45 degrees.

The peripheral cutting portions 16c can each include the base 22 and a sidewall 24c. As the base 22 of the third bit 12c is substantially similar to the base 22 of the drill bit 12, the base 22 associated with the drill bit 12c will not be described in detail herein. The sidewall 24c can be coupled to or integrally formed with the base 22. The sidewall 24c can include a first end 26c and a second end 28c. The first end 26c can define a tapered surface 30c to facilitate the channeling of the cut sections of the anatomy. The second end 28c can include a first cutting surface 38c. The first cutting surface 38c can generally be formed with a shallow cutting angle A3 so that the first cutting surface 38c can remove only a small amount of the anatomy for each revolution of the drill bit 12c. The shallow cutting angle A3 can range from about 0.01 to 65 degrees and generally from about 0.01 to 10 degrees. The first cutting surface 38c can be used to guide the drill bit 12c into the anatomy, as the first cutting surface 38c can extend above a surface R of the second end 28c. The first cutting surface 38c can be coupled to or integrally formed with the central cutting portion 18 to form a uniform cutting surface 45c.

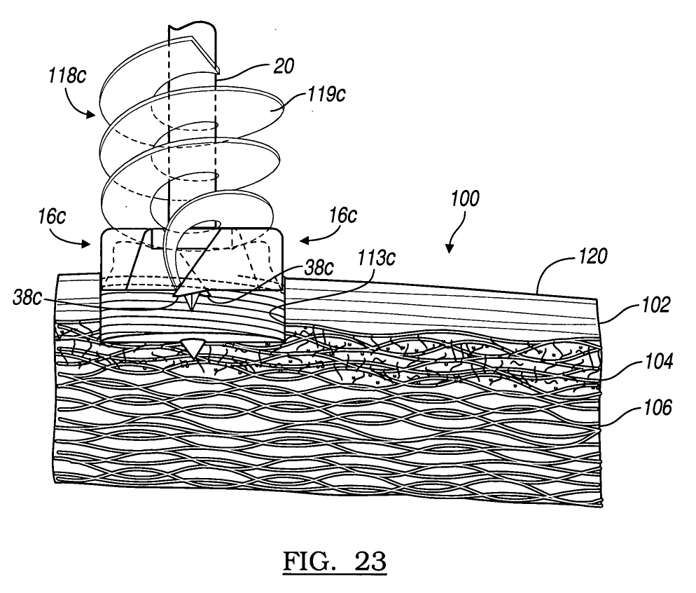

With additional reference now to FIGS. 22 and 23, wherein the same reference numerals denote the same or similar components, the anatomy 100 can include the cartilage layer 102, the cortical bone layer 104 and the cancelleous bone layer 106. The anatomy 100 can include the concave surface 120, such as the distal end of a femur. The drill bit 12c can be used to form a bore 112c in the concave surface 120, for receipt of the graft 110. Generally, the graft 110 can be used since the bottom surface 116 of the bore 112c formed by the uniform cutting surface 45 of the drill bit 12c is generally planar. Then, the drill bit 12c can be coupled to the drill 14. Next, the drill bit 12c can be positioned adjacent to the anatomy 100 and the drill 14 can be actuated such that the drill bit 12c begins to rotate. As the drill bit 12c begins to rotate, the first cutting surface 38c can guide the drill bit 12c into the anatomy 100.

As the drill bit 12c rotates, the uniform cutting surface 45 can remove thin layers 119c of the cartilage layer 102 of the anatomy 100, while the first cutting surfaces 38c can cut a perimeter 108 of the bore 112c in the cartilage layer 102. Generally, the thickness of the layer 119c removed per revolution of the drill bit 12c is between 0.1 mm and 0.5 mm. Simultaneously, the central cutting surfaces 42 can cut an interior of the bore 112c in the cartilage layer 102. The thin layers 119c of the cartilage layer 102 can pass into the angular channel 17c of the drill bit 12c to remove the cartilage layer 102 from the uniform cutting surface 45c. The shallow cutting angles A3 of the first cutting surfaces 38c can enable the drill bit 12c to remove the thin layers 119c of the anatomy 100 by a shallow cut made during each rotation of the drill bit 12c, which can be less traumatic to tissue surrounding the bore 112c, as shown by the cutting path 118c of the drill bit 12c. After the bore 112c has been formed with the desired depth D, the drill bit 12c can be removed and the graft 110 (or graft substitute) can be inserted into the bore 112c (similar to that shown in FIG. 6).

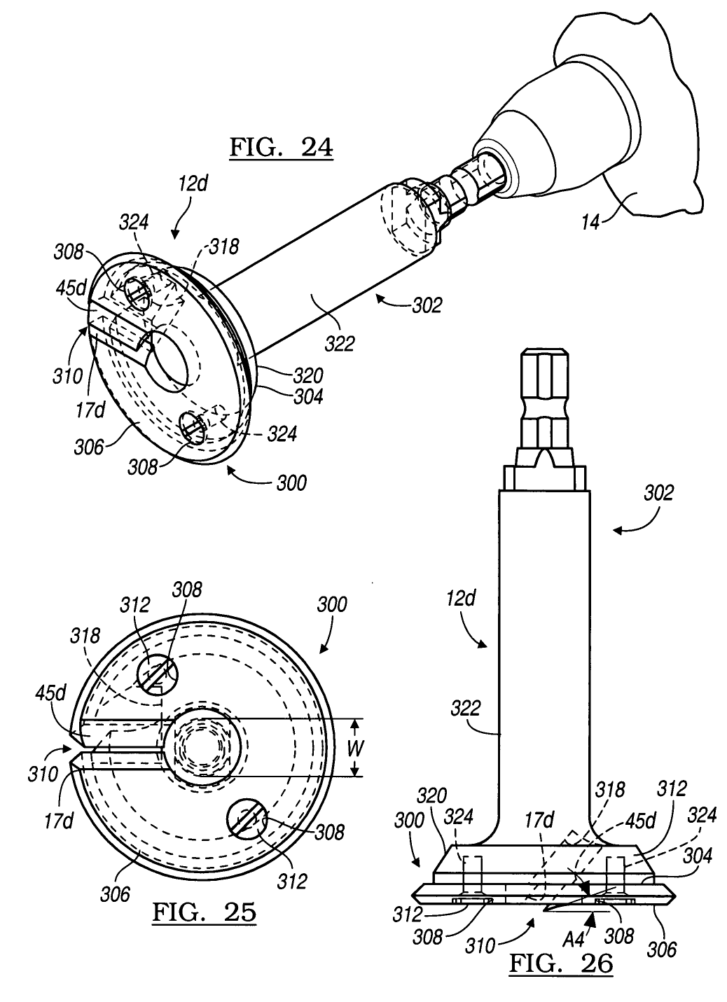

With reference now to FIGS. 24, 25 and 26, a fourth alternative drill bit 12d is shown. The drill bit 12d can include a planer blade 300 and a shaft 302. The drill bit 12d can form a flat bottomed bore in a portion of the anatomy, as will be discussed herein. The planer blade 300 can be coupled to the shaft 302.

The planer blade 300 can include a first surface 304, a second surface 306, and at least one or a plurality of throughbores 308 defined in the planer blade 300. The planer blade 300 can also include a cutting region 310. The first surface 304 of the planer blade 300 can generally be disposed opposite the second surface 306 and can be configured to couple the planer blade 300 to the shaft 302. The second surface 306 can be generally smooth to interface with the anatomy 100. The throughbores 308 can be adapted to receive a mechanical fastener, such as a screw 312, to couple the planer blade 300 to the shaft 302. It should be understood, however, that although the planer blade 300 is shown as mechanically coupled to the shaft 302, the planer blade 300 could be integrally formed with the shaft 302, or could be coupled to the shaft 302 by any other suitable process, such as welding and/or adhesives.

The cutting region 310 of the planer blade 300 can include a uniform cutting surface 45d and angular channel 17d. The uniform cutting surface 45d can extend beyond the second surface 306 of the planer blade 300. The uniform cutting surface 45d can have a shallow cutting angle A4 to facilitate the removal of only a small amount of the anatomy for each revolution of the drill bit 12d. The shallow cutting angle A4 can range from about 0.01 to 65 degrees and generally from about 20 to 30 degrees. The angular channel 17d can be formed adjacent to the cutting surface 45d and can have a width W. The angular channel 17d can be sized to facilitate the removal of thin layers 119d created during the rotation of the drill bit 12d. The angular channel 17d of the planer blade 300 is generally aligned with an angular channel 318 defined in the shaft 302.

The shaft 302 can define a base 320 and a stem 322. The base 320 can define the angular channel 318 and can define corresponding apertures 324 for receipt of the screws 312 to couple the planer blade 300 to the shaft 302. The stem 322 can be configured to be coupled to the drill 14. The stem 322 can be generally hollow to enable a portion of the drill 14 to pass therethrough. Further detail regarding the drill bit 12d is outside the scope of the present disclosure but an exemplary drill bit 12d is disclosed in greater detail in commonly assigned United States patent entitled “Bone Face Cutter,” filed on Aug. 11, 1992, U.S. Pat. No. 5,336,226, which is incorporated by reference herein in its entirety.

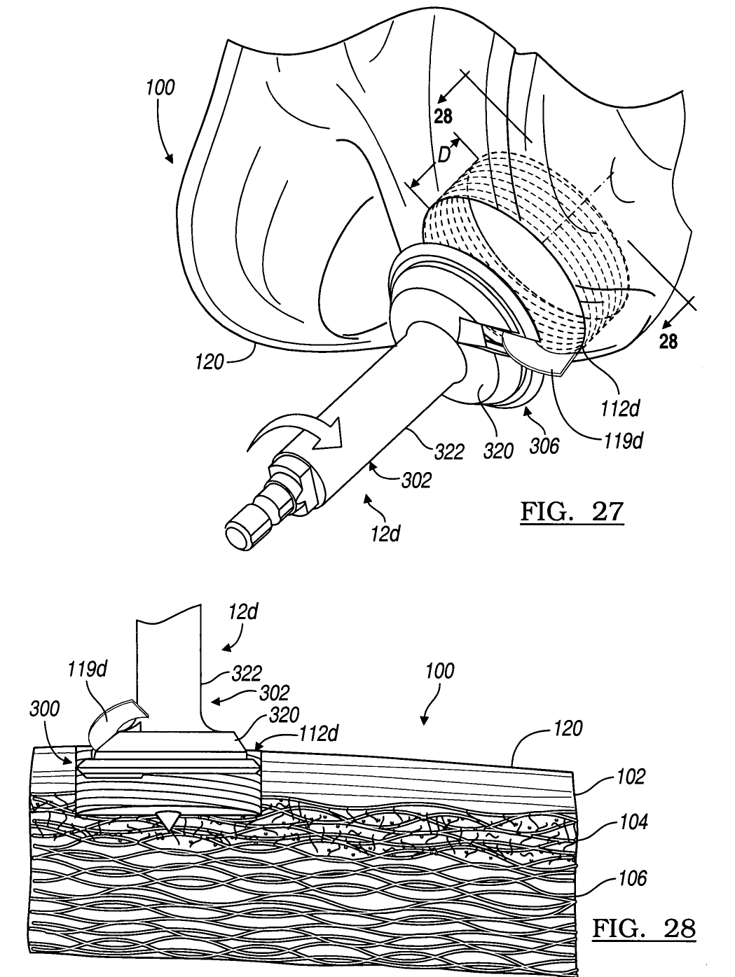

With additional reference now to FIGS. 27 and 28, wherein the same reference numerals denote the same or similar components, the anatomy 100 can include the cartilage layer 102, the cortical bone layer 104 and the cancelleous bone layer 106. In addition, the anatomy 100 can include the convex surface 120, such as the distal end of a femur. The drill bit 12d can be used to prepare the anatomy 100 for receipt of the graft 110 by forming a bore 112d in the anatomy 100.

In order to form the bore 112d in the concave surface 120, for receipt of the graft 110, the planer blade 300 can be coupled to the shaft 302. Once assembled, the drill bit 12d can be coupled to the drill 14 via the shaft 302. Then, the drill bit 12d can be positioned adjacent to the anatomy 100 and the drill 14 can be actuated.

As the drill bit 12d rotates, the uniform cutting surface 45d can remove thin layers 119d of the cartilage layer 102 of the anatomy 100 to form the bore 112d. Generally, the thickness of the layer 119d removed per revolution of the drill bit 12d is between 0.1 mm and 0.5 mm. The thin layers 119d created during the cutting process can enter the angular channels 17d, 318. The shallow cutting angle A4 of the uniform cutting surface 45d can enable the drill bit 12d to remove the thin layers 119d of the anatomy 100 by a shallow cut made during each rotation of the drill bit 12d, which can be less traumatic to tissue surrounding the bore 112d.

Once the drill bit 12d has created the bore 112d with a desired depth D, which could include removing a portion of either the cortical bone layer 104 and/or the cancelleous bone layer 106, the drill bit 12d can be removed and the graft 110 (or graft substitute) can be inserted into the bore 112d as shown in FIG. 6.

The description of the teachings herein is merely exemplary in nature and, thus, variations that do not depart from the gist of the teachings are intended to be within the scope of the teachings. Such variations are not to be regarded as a departure from the spirit and scope of the teachings.

What is claimed is:

1. A method of preparing a portion of an anatomy for receipt of a graft comprising:
providing a cutting tool having a solid, shallow angle cutting surface; and
cutting a shallow section out of the anatomy with the solid, shallow angle cutting surface to form a bore.

2. The method of claim 1, further comprising:
inserting the graft into the bore.

3. The method of claim 1, further comprising:
forming the bore with a bottom surface having a contour substantially similar to a contour of the cutting tool.

4. The method of claim 3, further comprising:
providing the cutting tool with a concave cutting surface; and
cutting a convex surface on a bottom of the bore.

5. The method of claim 3, further comprising:
providing the cutting tool with a convex cutting surface; and
cutting a concave surface on a bottom of the bore.

6. The method of claim 3, further comprising:
providing the cutting tool with a flat cutting surface; and
cutting a flat surface on a bottom of the bore.

7. The method of claim 1, wherein providing the cutting tool with a solid, shallow angle cutting surface further comprises providing the cutting tool with a cutting surface having a cutting angle of between 10 and 45 degrees.

8. The method of claim 1, further comprising:
wherein cutting a shallow section out of the anatomy further comprises cutting approximately 0.1 mm to 0.5 mm (unit) of the anatomy per revolution of the cutting tool.

9. The method of claim 1, further comprising:
providing the cutting tool with at least one peripheral cutting surface; and
guiding the peripheral cutting surface into the anatomy.

10. A method of preparing a portion of an anatomy for receipt of a graft comprising:
providing a cutting tool having a solid, shallow angle cutting surface;
providing a graft or graft substitute;
cutting a shallow section out of the anatomy with the solid, shallow angle cutting surface to form a bore; and
inserting the graft into the bore.

11. The method of claim 10 further comprising:
selecting the graft from the group comprising: an osteochandral graft, a bone substitute material, a xenograft or combinations thereof.

12. The method of claim 11, further comprising:
selecting the bone substitute material from the group comprising:
polylactide (PLA), polyglycolic acid (PGA), Calcium Phosphate, Calcium Sulfate TriCalcuim Phosphate or combinations thereof.

13. The method of claim 10, further comprising:
providing the cutting tool with at least one peripheral cutting surface; and
guiding the peripheral cutting surface into the anatomy.

14. The method of claim 10, further comprising:
forming the bore with a bottom surface having a contour substantially similar to a contour of the cutting tool.

15. The method of claim 14, further comprising:
providing the cutting tool with a cutting surface selected from the group comprising a concave cutting surface, a convex cutting surface, and a flat cutting surface.

16. The method of claim 14, wherein providing the cutting tool with a solid, shallow angle cutting surface further comprises providing the cutting tool with a cutting surface having a cutting angle of between 10 to 45 degrees.

17. The method of claim 10, further comprising:
wherein cutting a shallow section out of the anatomy further comprises cutting approximately 0.1 mm to 0.5 mm (unit) of the anatomy per revolution of the cutting tool.

18. A method of preparing a portion of an anatomy for receipt of a graft comprising:
providing a cutting tool having a solid, shallow angle cutting surface, the shallow angle of the cutting surface ranging from 10 to 45 degrees, and a central cutting surface with a defined cutting surface, the defined cutting surface selected from the group comprising a flat cutting surface, a concave cutting surface, and a convex cutting surface;
providing a graft;
cutting a shallow section out of the anatomy with the solid, shallow angle cutting surface;
forming a bore with a contoured bottom surface, the contour of the bottom surface being substantially similar to the defined cutting surface of the central cutting surface; and
inserting the graft into the bore.

19. The method of claim 18, further comprising:
providing the cutting tool with at least one peripheral cutting surface; and
guiding the peripheral cutting surface into the anatomy.

20. The method of claim 18, further comprising:
wherein cutting a shallow section out of the anatomy further comprises cutting approximately 0.1 mm to 0.5 mm (unit) of the anatomy per revolution of the cutting tool.
